(19) United States
(12) Patent Application Publication
(10) Pub. No.: US 2018/0214306 A1
(43) Pub. Date: Aug. 2, 2018

(54) OPHTHALMIC SURGICAL APPARATUS

(71) Applicants: ALPHANOV CENTRE TECHNOLOGIQUE OPTIQUE ET LASERS, Talence (FR); CENTRE HOSPITALIER UNIVERSITAIRE DE BORDEAUX, Talence (FR); MORIA LASE, Antony (FR)

(72) Inventors: Bruno CHASSAGNE, Pessac (FR); Florent DELOISON, Bordeaux (FR); David TOUBOUL, Bordeaux (FR); Francois SALIN, Gradignan (FR); Pierre DESLANDES, Gradignan (FR)

(21) Appl. No.: 15/562,125

(22) PCT Filed: Apr. 1, 2016
(86) PCT No.: PCT/FR2016/050754

§ 371 Date: Jan. 10, 2018

(30) Foreign Application Priority Data
Apr. 1, 2015 (FR) 1552791

(51) Int. Cl. A61F 9/008

(52) U.S. Cl. CPC A61F 9/0084; A61F 2009/0087; A61F 2009/00897; A61F 2009/00887

(57) ABSTRACT

An ophthalmic surgical apparatus includes: a laser source emitting a beam of one picosecond to one nanosecond pulses; an optical system focusing the beam on a focal point; and a system for moving the focal point along a predetermined path. The laser source generates the beam, focused next to a surface of the anterior segment of the eye, the focal point located at a distance from an optical axis of symmetry of the anterior segment of the eye. The system for moving the beam includes a single degree of freedom of rotation about an axis of rotation to move the focal point along a curved path located in an annular area about the optical axis of symmetry of the anterior segment of the eye. The ophthalmic surgical apparatus has a numerical aperture limiting the geometric optical aberrations at the focal point and on the entire curved path.

### TECHNICAL FIELD

This invention relates to ophthalmic surgical apparatuses. More precisely, the invention relates to a surgical apparatus of the anterior segment of the eye. In particular, the invention relates to a surgical apparatus for the treatment of cataract.

### PRIOR ART

Cataract is a disease of the eye, mainly linked to age, which every year afflicts hundreds of thousands of people in the world. Cataract produces a progressive opacification of the crystalline lens. The crystalline lens is an optical medium, normally transparent, of the eye that has the form of a biconvex lens between the cornea and the retina. The crystalline lens comprises a capsule, also referred to as crystalline lens bag, and a core placed at the centre of the capsule. The capsule is connected by ligaments to muscles that make it possible to modify the curvature of the crystalline lens. The crystalline lens as such allows for the accommodation, i.e. the formation of images on the retina according to the distance of vision.

Surgical treatment of the cataract is without a doubt the most practiced microsurgical act in the world. This treatment generally consists in extracting the crystalline lens or a portion of the opacified crystalline lens, and in replacing it with a synthetic crystalline lens implant.

A first type of surgical treatment is based on the use of conventional surgical tools, such as scalpels and on a phacoemulsification probe. This conventional technique requires long learning of the gesture of the surgeon and a high level of expertise in order to obtain satisfactory results.

A conventional cataract operation can be broken down into several steps, carried out by means of one or several manual tools. A cutting tool, for example a scalpel, is used to form one or two mini-incisions, in general less than 2 mm long, at the periphery of the cornea, in order to allow for the introduction of the other surgical instruments as close as possible to the crystalline lens. The step of capsulorhexis, or circular capsulotomy, consists in making a circular or curvilinear cut of the anterior capsule of the crystalline lens. This cut is conventionally carried out manually by means of special forceps. The diameter of the capsulorhexis is in principle 5.5 mm. During a manual cut, the exact diameter of this capsulorhexis can be difficult to control and good circularity is difficult to obtain. This step of capsulorhexis conditions the safety of the following step of extracting the core from the crystalline lens fragmented by ultrasound. To this effect, an ultrasound phacoemulsification probe is introduced inside the crystalline lens capsule in order to fragment the core. A suction system withdraws the fragments of the core. Then an intraocular crystalline lens implant is installed in the posterior portion of the capsule. The circularity of the cut and its precise diameter are very important elements in the precise positioning of the implant in particular for the new multifocal implants called premium implants.

This technique has benefitted from technological advances concerning phaco-emulsificators and intraocular implants.

This technique applies not only to treatment of the cataract, but also to refractive surgery of the crystalline lens. Indeed, special implants exist, referred to as premium implants, which make it possible to correct certain sight defects such as astigmatism, presbyopia, hyperopia or myopia.

A second type of surgical treatment of the eye is based on the use of femtosecond lasers.

Femtosecond lasers are commonly used in ophthalmic surgery in the LASIK technique of cutting the cornea in the treatment of myopia.

Cataract surgical apparatuses based on a femtosecond laser have appeared more recently. A femtosecond laser is a laser that emits pulses with a duration between 1 and a few hundred femtoseconds. Femtosecond lasers emit ultra-short high-power pulses, which allow for the cutting of ocular tissues without local heating. Coupled to a three-dimensional imaging system and to a robotic system for moving with micrometric precision, a femtosecond laser makes it possible to assist, optimise and secure the exeresis surgery of the crystalline lens. An ophthalmic surgical system using a femtosecond laser guarantees a precision of the centring and a reproducibility of the capsulorhexis diameter that is clearly superior to those obtained by manual operations.

In femtosecond laser-assisted cataract surgery (FLAC), the femtosecond laser makes it possible to make the cut of the anterior capsule of the crystalline lens according to a pre-established path, often circular, and a fragmentation of the crystalline lens core. However, in certain particular cases, it is observed that the successive laser impacts can produce a cut of which the edge has a toothed aspect (or as a postage stamp) due to the focussing of the laser beam and to the spatial shift of the beam between the impacts of the femtosecond laser.

Certain femtosecond lasers also make it possible to carry out corneal incisions intended for the passage of surgical instruments or the carrying out of corneal limbic incisions aimed at treating refractive errors, such as astigmatism. Such a femtosecond laser is in general coupled to a phacoemulsification probe which fragments the core into fragments that are small enough to be sucked via a probe.

The FLAC technique theoretically makes it possible to direct the energy of the laser in an extremely focussed manner. However, this focussing of the laser beam is in practice limited by the presence of optical aberrations and/or diffusion due to the optical media passed through, for example in the case of so-called “white” cataracts.

In addition, the FLAC technique requires prior examination via imaging of the dimensions and positions of the cornea, of the iris and of the thickness of the crystalline lens. This information is essential in determining the position of the focal point of the laser beam in three dimensions in order to avoid damaging the posterior face of the capsule or posterior capsule. However this analysis requires the implementation of a special three-dimensional imagery apparatus and the processing of the acquired images currently takes several minutes. Once the acquisition and the three-dimensional image processing are complete, the surgeon validates the target marks of the laser and triggers the laser. During these two operations, the laser must remain coupled to the eye of the patient via a complex eye/machine adaptation interface. The eye is immobilised beforehand and the pupil is dilated by injection of drops on the eye. The deferred image processing does not allow for a real-time control of the movements of the eye or of the pupil, which can cause difficulties in case of uncontrolled movement of the eye or unexpected contraction of the pupil. In addition the dimension and the rigidity itself of the machine to which the system for coupling to the eye is attached does not allow for a flexible and fast movement of this machine with respect to the eye.

Finally, the cost of femtosecond laser-assisted cataract surgery systems remains very high, without any notable reduction in the duration of the surgery.

### Technical Problem

There is therefore a need for an ophthalmic surgical system, applied in particular to cataract treatment, that makes it possible to improve the quality and the safety of ophthalmic surgical systems while still reducing the duration of an ophthalmic surgery and the cost of such surgery.

This invention has for purpose to overcome these disadvantages and relates to an ophthalmic surgical apparatus comprising a laser source suitable for emitting a beam of laser pulses, an optical focussing system arranged on the optical path of the pulsed laser beam, with the optical focussing system being suited for focussing said beam of laser pulses on a focal point intended to be positioned on a portion of the anterior segment of an eye and a system for moving the beam of laser pulses configured to move said focal point along a predetermined path.

According to the invention, preferably, the laser source generates a pulsed laser beam that has a duration of about one picosecond to one nanosecond, the optical focussing system is configured to focus the pulsed laser beam on a focal point in the vicinity of a surface of the anterior segment of the eye, the focal point being located at a distance d other than zero from an optical axis of symmetry of the anterior segment of the eye, the system for moving the beam of laser pulses includes a single degree of freedom of rotation about an axis of rotation substantially parallel to the optical axis of symmetry of the anterior segment so as to move said focal point along a curved path located in an annular area about the optical axis of symmetry of the anterior segment of the eye and the optical focussing system is configured, for example by means of a limited numerical aperture, so as to limit the geometric optical aberrations at the focal point and on the entire curved path in said annular area about the optical axis of symmetry of the anterior segment of the eye.

As such, the ophthalmic surgical apparatus allows for a circular cut for example of the anterior capsule of the crystalline lens. The cut is very fast, as it entails only a single movement of rotation. The quality of this cut is excellent due to the limitation of the optical field to a single focussing point on the entire curved path which greatly facilitates the correction of optical aberrations. This apparatus in addition allows the operator or the surgeon to perform, via a binocular microscope, a real-time control of the proper unfolding of the surgery.

Particularly advantageously, the system for moving the pulsed laser beam comprises an optical system, arranged on an optical path of the laser beam upstream or downstream of the lens or of the focussing mirror, the optical system being suited to receive the incident laser beam and configured to form an angularly deflected or translated laser beam with respect to the incident laser beam, and wherein said optical system comprises at least one optical component mounted mobile in rotation about said axis of rotation in such a way as to produce a rotation of the laser beam.

According to another embodiment, the system for moving the pulsed laser beam comprises a prism arranged on an optical path of the pulsed laser beam, said prism being mounted mobile in rotation about an axis of rotation.

According to another embodiment, the system for moving the pulsed laser beam comprises at least one mirror arranged on an optical path of the pulsed laser beam, in such a way as to induce an angular deviation and/or a lateral shift of the pulsed laser beam, and said at least one mirror being mounted mobile in rotation about an axis of rotation.

Advantageously, the system for moving the pulsed laser beam is configured to move said focal point along a circular path with a determined radius.

According to a particular and advantageous aspect of the invention, the system for moving the pulsed laser beam further comprises a degree of freedom of translation along an axis of translation parallel to the axis of rotation, and the system for moving is configured to move said focal point along a helical path of circular section and with a determined radius.

Alternatively, the curvilinear path is of elliptical section, and of determined and possibly variable dimensions.

Particularly advantageously, the ophthalmic surgical apparatus comprises on the one hand, a manual tool comprising the optical focussing system and the system for moving the pulsed laser beam, and, on the other hand, a fibre optic link arranged between the laser source and the manual tool.

As such, the ophthalmic surgical apparatus can be adjusted quickly in position and in angle with respect to the optical axis of the eye by the single hand of the surgeon in such a way as to move, in position and/or in angle, the curvilinear path inside the anterior segment of the eye by directly moving the manual tool located at the end of the optical fibre and only this manual tool, which as such forms a genuine surgical instrument.

Preferably, the manual tool comprises a semi-reflective mirror or a dichroic mirror arranged on the optical path of the laser beam and wherein the manual tool is adapted for optically combining a binocular microscope in such a way as to provide a visual inspection in real time of the anterior segment of the eye.

In particular, this fibre optic link between the laser and the system for focussing can be flexible and wired, allowing for an offset of the laser source. A fibre optic link furthermore allows for the flexibility of the system for focussing which can then be integrated into a manual tool that can in particular be directly held in the hand by the operator.

According to a particular aspect of the invention, the ophthalmic surgical apparatus further comprises an adaptation interface device that comprises a lame with planar and parallel faces and/or a plano-concave plate, with the adaptation interface device having at least one optical surface configured in such a way as to correct the optical aberrations at the focal point and on said path of said focal point. Optionally, the device can comprise a system suitable for exercising a low-pressure suction on the eye.

As such, the ophthalmic surgical apparatus can be arranged on the eye to be treated, with the adaptation interface device being on the eyeball of the eye.

Advantageously, the ophthalmic surgical apparatus further comprises a device for triggering firings of the laser source and of the system for moving the pulsed laser beam.

In an embodiment, the laser source emits laser pulses at a wavelength between 700 nm and 1350 nm, preferably between 1025 nm and 1080 nm.

Advantageously, the laser source emits laser pulses at a repetition rate between 20 kHz and 1 MHz, and preferably greater than or equal to 240 kHz.

According to an embodiment, the ideally pulsed single-transverse mode laser source is suitable for emitting a beam of laser pulses with a duration between 1 picosecond and 100 ps. Particularly advantageously, the ideally pulsed single mode laser source comprises a semiconductor laser or other laser suited for emitting a beam of laser pulses with a duration of between one picosecond and 30 ps. Optimally the duration (measured at mid-height of their time profile) of the pulses at the focal point is between 1 ps and 5 ps.

According to another embodiment, the ideally pulsed single-transverse mode laser source is suitable for emitting a beam of laser pulses with a duration of between 0.1 nanosecond and 10 ns.

The invention will have a particularly advantageous application in an ophthalmic surgical apparatus of the anterior segment of the eye.

This invention also relates to the characteristics that will appear in the following description and which must be considered separately or according to all technically permissible combinations thereof.

This description given by way of a non-limiting example will make better understood how the invention can be carried out in reference to the annexed drawings wherein:

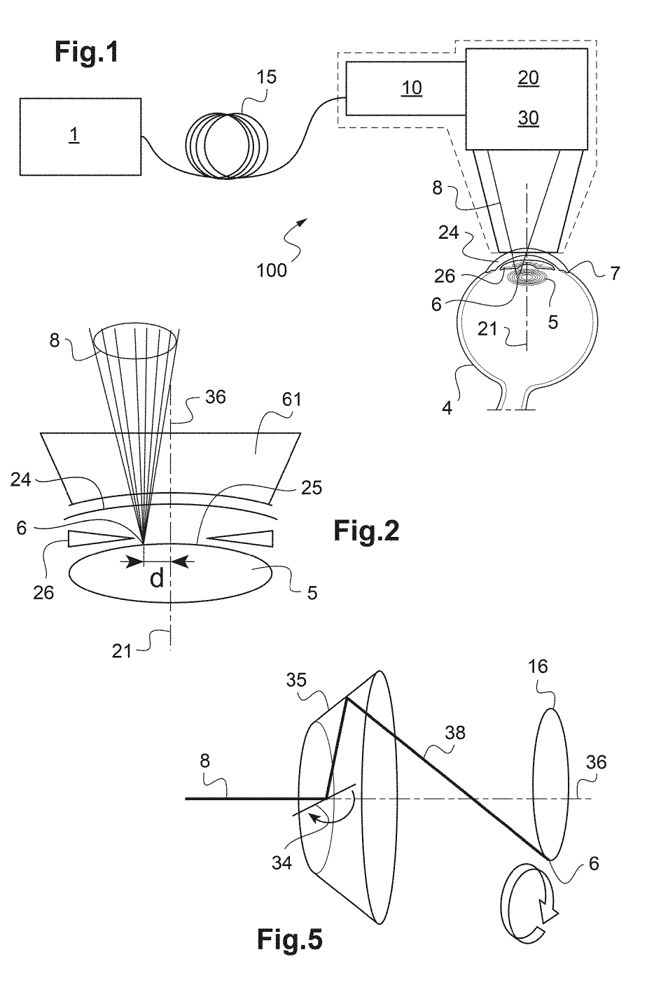

FIG. 1 diagrammatically shows a general view of an ophthalmic surgical apparatus according to an embodiment of the invention;

FIG. 2 diagrammatically shows a cross-section view of an adaptation interface device between the laser system and the eye to be treated;

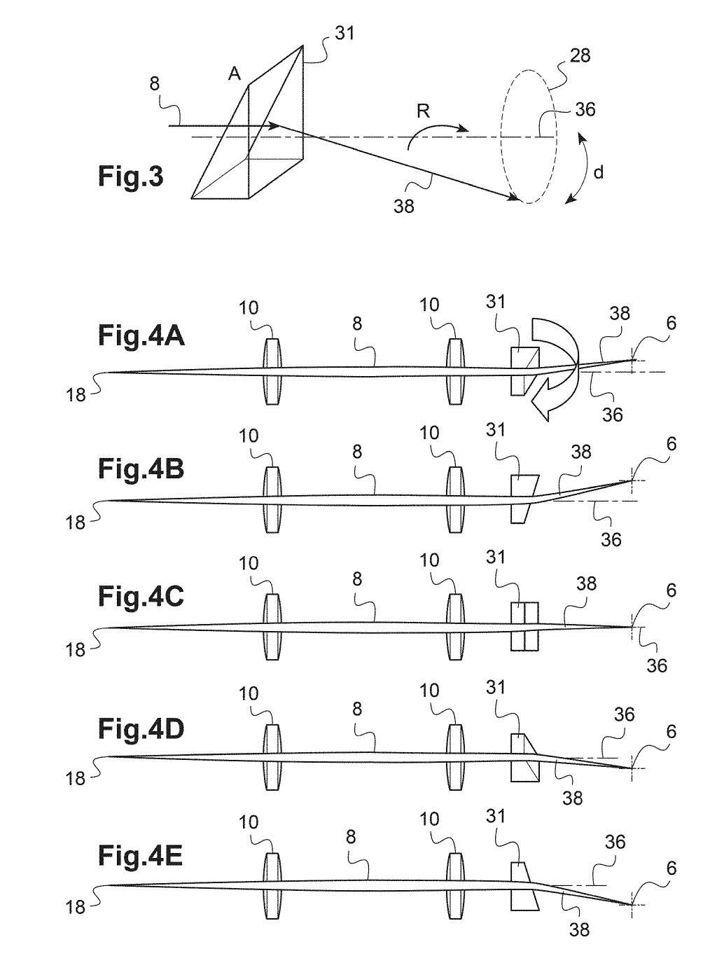

FIG. 3 diagrammatically shows a first embodiment of a system for moving the laser beam based on a rotating prism;

FIGS. 4A-4E show the combination of an optical focussing system and of a rotating prism in different orientations of the prism and the corresponding positions of the focal point;

FIG. 5 diagrammatically shows a second embodiment of a system for moving the laser beam based on a system of mirrors comprising a rotating mirror;

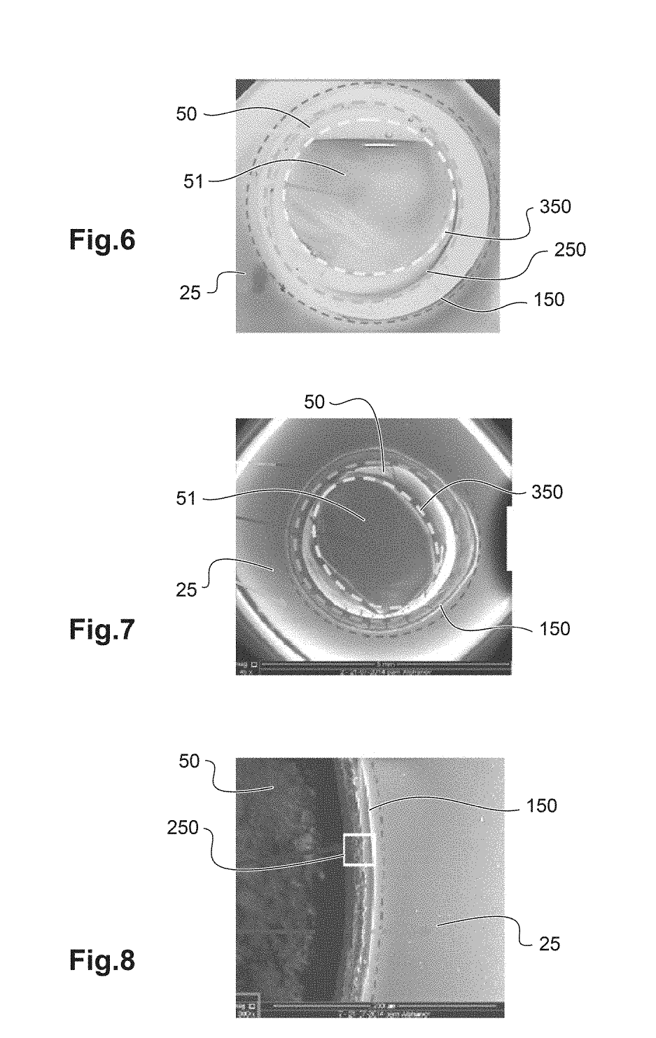

FIGS. 6 and 7 show examples of images taken by binocular microscopy after capsulorhexis surgery carried out by means of an apparatus according to an embodiment of the invention;

FIG. 8 shows an example of a scanning electron microscopy image that shows the edges of a rhexis in the crystalline lens capsule.

### DETAILED DESCRIPTION

Device

Many cornea surgical apparatuses, of the LASIK type, or cataract surgical apparatuses (FLAC) are based on a femtosecond laser. The term femtosecond laser here means a laser that emits light pulses with a duration between one and a few hundred femtoseconds. Minimising the duration of the pulses is generally recommended for cutting transparent tissues of the anterior segment of the eye. Indeed, the longer the duration of the laser pulses laser is, the greater the deposit of energy is and risks generating thermal effects. It is essential to minimise the deposit of energy and to avoid a heating of the ocular tissues able to cause irremediable damage to them.

An observation that is part of this invention is that all of the systems that use a femtosecond laser for cataract surgery are based on a device for moving the beam configured to make it possible to focus the beam on any point of a volume that corresponds to a very large portion of the crystalline lens.

These prior art systems use, on the one hand, a mechanical system for moving the focal point with six degrees of freedom (three degrees of freedom of rotation and three degrees of freedom of translation) and, on the other hand, a three-dimensional optical imaging system. However, it is very difficult and even impossible to obtain a focussing that is free of geometric optical aberrations on an image field as extended as the volume of the crystalline lens. Complex optical systems can be used to attempt to compensate the optical aberrations but it can easily be shown that it is in practice impossible to perfectly compensate all of the optical aberrations on a field with a variable diameter.

In addition the method used in these laser systems of prior art requires an immobilisation of the eye for a duration far greater than a second, and in any case greater than the duration during which a patient can keep his eye immobile. All of the prior femtosecond laser-based systems therefore use an adaptation interface device that applies a pressure by suction that is sufficient to immobilise the eye during the 3D image acquisition and during the cataract surgery. Consequently, the immobilisation of the eye in practice lasts from several dozen seconds up to several minutes. However, the suction pressure exerted on the eye is known to induce many secondary effects including haemorrhages, a detrimental increase of the intraocular pressure or, in certain cases, the appearance of ulcers.

This disclosure proposes an ophthalmic surgical apparatus dedicated in particular to the cutting of the anterior capsule of the crystalline lens also referred to as capsulorhexis.

On the one hand, this apparatus is based on the use of a pulse laser preferably with a duration of one picosecond or nanosecond, instead of a femtosecond laser. The apparatus can also operate with a femtosecond laser, but the apparatus is then more expensive.

The term picosecond laser here means a laser that emits light pulses of a duration between 0.1 picosecond and about 100 ps. Finally, the term nanosecond laser means a laser that emits light pulses with a duration between 0.1 nanoseconds and about 100 ns.

The laser 1 is preferably a single-transverse mode laser.

On the other hand, according to this disclosure, the system for moving the laser beam is limited to a system that has a single degree of freedom of rotation. Optionally, the system for moving the laser beam can have one, two or three degrees of freedom in translation, of a limited amplitude. As such, the movement of the laser beam is limited to a curvilinear path located in a restricted volume, preferably of an annular or toric shape. The mechanical system for moving is extremely simplified and the cost of the apparatus is reduced. In addition, limiting the path of the focussing point to a circle (which optically corresponds to a field limited to a single point) makes it possible to correct the optical aberrations at the focal point over the entire path of the laser beam because if positioned in the referential of the focal point the element in rotation is immobile. Finally, limiting the path to a restricted volume makes it possible to suppress the need for a three-dimensional imaging system. A two-dimensional imaging system of the binocular microscope type such as those conventionally present in the operating room suffice for monitoring and controlling the focussing of the laser beam in real time over the entire path.

In particular, by limiting the path of the focal point to a circle centred on the optical axis of the laser beam before the deflection thereof by a prism or an off centered focussing lens for example, it is possible to have at any point of the path exactly the same wavefront. It is then particularly easy to correct the wavefront since the correction at one-point results in the same corrections for all of the points with the conditions of rotating the element for deviating or deflecting the laser about the optical axis of the laser before deviation.

All of the elements located on the path of the beam after deviation can advantageously be with symmetry of revolution with respect to the axis of the laser on any point of the path of the latter when it has an interface with a significant change in index. Such a surface located after the focussing lens for example can have the shape of a conical frustum of which the angle is such that the main radius is always perpendicular to the incident surface at any point of its path.

FIG. 1 diagrammatically shows an ophthalmic surgical apparatus 100 according to an embodiment of the invention. The apparatus is arranged with respect to an eye 4 for a surgery of cutting the anterior capsule of the crystalline lens. A cross-section view is diagrammatically shown of the eye 4 of a patient that shows a few anatomical elements of the eye 4: the cornea 24, the limbus 7 around the cornea, the iris 26 and the crystalline lens 5. In general, during capsulorhexis surgery, the iris 26 is dilated to the maximum. An optical axis 21 of symmetry is defined of the crystalline lens as being the axis that passes through the centre of the iris 26 or the centre of the limbus 7 or a point located between these two centres and with this optical axis 21 being substantially perpendicular to the surface of the anterior capsule of the crystalline lens.

The ophthalmic surgical apparatus comprises a laser source 1 connected to preferably by optical fibre 15 to a manual tool 40. The optical fibre 15 allows for an easy manipulation of the manual tool, while still leaving the laser source 1 fixed and at a distance from the patient. The optical fibre makes it possible as such to clear the space around the eye 4 of the patient. An operator, or a surgeon, places the manual tool 40 in the vicinity or in contact with the cornea 24 of the eye 4 of the patient.

The laser 1 is advantageously a picosecond or nanosecond pulse laser. Such a laser is compatible with the transmission via an optical fibre 15, contrary to a femtosecond laser which emits a pulse power that can destroy the optical fibre 15.

The manual tool 40 comprises an optical system 10 for shaping the laser beam and an optical focussing system 20 for focussing the laser beam 8 on an intraocular focal point 6 and more precisely on a point of the anterior segment of the eye 4 of the patient.

The optical system 10, 20 comprises for example one or two afocal optical systems with lenses. The optical focussing system 20 is configured to focus the focal point 6 in the vicinity of the surface of the anterior capsule of the crystalline lens and in such a way that the focal point 6 of the laser beam 8 is off centered with respect to the optical axis 21 of symmetry of the crystalline lens. As such, the incident laser beam 8 on the eye propagates through various optical media outside the axis of the anterior segment of the eye. More precisely, the laser beam 8 is refracted by a portion outside of the axis of the cornea 24 and transmitted through the aqueous humour located between the posterior face of the cornea and the anterior capsule of the crystalline lens 5.

The manual tool 40 also comprises a system for moving 30 the laser beam 8 suitable for moving the focal point 6 with respect to an axis of rotation. More particularly, the system for moving 30 the focal point 6 of the laser beam is configured to constrain the focal point 6 to follow a curvilinear path 16 about an axis of rotation. Preferably, the surgeon arranges the manual tool 40 in such a way as to align the axis of rotation on the optical axis 21 of symmetry of the crystalline lens. It is supposed here that the eye 4 remains fixed, without necessarily being immobilised. Particularly advantageously, the path 16 of the focal point 6 of the laser beam 8 is located on the surface of a cylinder or on a helicoid having an axial symmetry, for example of elliptical or circular section and with determined dimensions or with a determined diameter, with the axis of the cylinder being centred on the optical axis 21 of symmetry of the crystalline lens.

In particular this path 16 can begin in the volume of the crystalline lens 5 and finish between the surface 25 of the anterior capsule of the crystalline lens and the cornea 24.

Advantageously, the manual tool 40 comprises an adaptation interface device 60 placed in contact with the eye to be treated, which makes it possible to reduce the angle of incidence of the beam 8 on the cornea 24. The eye of the patient can be free or immobilised for a short duration (in general less than one second) by means of a weak suction. The manual tool 40 to which the adaptation interface device 60 is fixed forms as such an ophthalmic surgical instrument connected by optical fibre to the laser source, which allows for easy manipulation by the surgeon.

In a particularly advantageous embodiment, the manual tool 40 also comprises a semi-transparent plate or a dichroic plate, arranged on the optical path of the laser beam 8 and which makes it possible to directly view the anterior capsule of the crystalline lens and the focal point 6 of the laser beam or to optically couple a binocular microscope on the optical path of the laser beam. Such a binocular microscope makes it possible to simultaneously view the anterior capsule of the crystalline lens and the focal point 6 of the laser beam. The binocular microscope allows as such for the real-time control of the alignment of the manual tool 40 in relation to the optical axis 21 of symmetry of the crystalline lens, the focussing of the laser beam 8 and the cutting of the anterior capsule of the crystalline lens. However, the direct viewing by the surgeon offers the advantage of allowing for a precise manual alignment of the ophthalmic surgical instrument in an extremely short time and the carrying out of the cutting of the rhexis in a total time of less than a few seconds or even one second.

FIG. 2 shows an enlarged cross-section view of a portion of an adaptation interface device installed in contact with the anterior portion of the eye of a patient. The adaptation interface device here comprises for example a plano-concave lens 61 of which the face arranged facing the cornea 24 has a radius of curvature greater than or equal to the average radius of curvature of the cornea 24. In another embodiment, the adaptation interface device comprises a plate with planar faces and parallel instead of the plano-concave lens 61. The adaptation interface device can be formed from a solid material or from a liquid material or from a combination of solid and liquid materials. These materials must be transparent to the wavelength of the laser. It is important to centre the optical axis of the adaptation interface device 60 on the optical axis 21 passing through the centre of the limbus and/or the centre of the iris. Advantageously, a liquid or a gel can be placed between the surface of the cornea 24 and the plano-concave lens 61 or the plate with planar faces of the adaptation interface device, in order to limit the deviation of the laser beam 8 by refraction on the interfaces between optical media with different refraction indexes.

Preferably, the lower surface of the interface 61 is spherical or quasi-spherical and of a radius of curvature that is slightly greater than that of the cornea, generally between 9 mm and 11 mm and preferably of 10 mm. As such, the contact between the ophthalmic surgical instrument and the eye is reduced to a single point or to a very small quasi planar surface which allows for a maximum lateral movement typically +/−0.5 mm to +/−1 mm in order to compensate an off centering of the iris with respect to the apex of the cornea while still maintaining an optical contact with the cornea. The adjusting of the alignment of the surgical instrument is carried out by the surgeon via a manual moving of the ophthalmic surgical instrument on the eye and not by movement of the laser beam inside the apparatus as is the case in systems based on the use of a scanning beam system of the scanner type. As such the laser beam describes a circle of which the position and the orientation in the eye can be adjusted by the surgeon by a simple modification of the angle and of the position of the ophthalmic surgical instrument on the surface of the eye in a time that is less than the time that is characteristic of movements of the eye. It is therefore not necessary to immobile the eye.

In the diagram of FIG. 2, the laser beam 8 successively passes through the plano-concave lens 61, the medium (air or liquid medium of index) located between the cornea 24 and the concave face of the lens 61, the cornea 24 and the aqueous humour present in the anterior chamber of the eye.

The laser beam 8 is focussed on a focal point 6. It is observed that the laser beam 8 passes through the lens 61 and the cornea off centered with respect to the optical axis of symmetry of revolution of these optical components, which is here merged with the optical axis 21. However, the numerical aperture of the laser beam 8 is limited to the extent that the area passed through by the laser beam 8 has a highly reduced spatial extent on the lens 61 and on the cornea 24.

The optical thickness of the plano-concave lens 61 or of the optical system forming the adaptation interface device in contact with the eye can be very high. In practice, the optical thickness of the adaptation interface device can reach 90% to 98% of the focal length of the optical focussing system. This thickness can even reach 100% in the case where the focussing element is not in movement or only makes slow movements as in the case where it is the prism that allows for the deviation for example. In the case where the immersed surface allows for the focussing and or also to correct the aberrations, a sufficient Δn must be maintained between the index of the focussing element and that of the immersion medium. The filling of the space that separates the optical focussing system of the eye with a medium with a refractive index greater than 1 and advantageously close to that of the cornea (of which the refractive index is approximately 1.38) makes it possible to increase the back focal distance, i.e. the distance from the focal point to the apex of a lens, for a given system for focussing without increasing the physical size of the focal spot. In addition, the fact that the optical focussing system combined with the adaptation interface device works on a single point of the field makes it possible to precisely compensate the geometric aberrations over the entire circular path including for a concave planar lens 61 of very great thickness.

In an embodiment, the adaptation interface device comprises a plano-concave lens or an optical system with several dioptres formed of a continuous assembly of lenses or of thick plates 61 comprised of several materials of which the adjacent surfaces coincide and of which the indexes are close to one another. Preferably that jump in the index Δn between two successive dioptres is less than 0.1. In addition, the materials are chosen to have a refractive index close to that of the cornea (n=1.38) typically between 1.3 and 1.5, so as to create an optically continuous thick assembly, i.e. without interface with the air except for the interface the farthest from the eye. Preferably the solid materials are chosen from melted silica (n=1.45) or low-index lenses (n<1.51) or polymers such as PMMA (n=1.49) or acrylic (n=1.49), and the liquid materials are chosen from water (n=1.33), from salted or sweet water (n=1.33 to 1.45) or gels with an aqueous base. One or several thick optical assembly interfaces can be formed from a liquid or a gel in order to retain optical continuity. As the optical focussing system works only on a single point of the optical field, it is possible to perfectly compensate the spherical aberration on this single point of the optical field despite the multitude of optical media passed through.

The focal point 6 of the laser beam 8 is positioned on a point on the surface of the anterior capsule of the crystalline lens 5, located at a determined distance d from the optical axis 21. For example, the distance d between the focal point 6 and the optical axis 21 is equal to 2.5 mm. Advantageously, the distance d can be adjusted according to the specific needs of a patient before the starting of the laser firing. For example, the distance d can be adjusted between 1 and 4 mm.

All of the optical components and optical media arranged on the optical path of the laser beam between the laser source 1 and the focal point 6 participate in the formation of the focal point 6. The combination of the optical components of the ophthalmic surgical apparatus with the portion of the anterior segment of the eye located between the anterior capsule and the anterior face of the cornea as such forms a complete optical system. More precisely, the optical systems 10, 20, the plano-convex lens 61 and the various optical media and interfaces of the eye located between the plano-convex lens 61 and the focal point 6 determine the position and the properties of the focal point 6 in terms of geometric optics.

The geometric optical performance of the complete optical system are easily limited by diffraction for high numerical apertures (N.A. at least equal to 0.4) and a fortiori for low numerical apertures (N.A. typically less than 0.2, preferably between 0.05 and 0.15 for example approximately 0.1), as the focussing in the image plane has a single field while still having a substantial working distance (for example working distance between the optical system of the manual tool 40 and the focal point 6 greater than 20 mm for a NA greater than or equal to 0.4).

In practice, the spatial extent of the laser beam 8 through the plano-concave lens 61 and the media of the anterior segment of the eye is very low (see FIG. 2). It is as such possible to reduce or even cancel the geometric aberrations at the focal point 6.

As indicated hereinabove, the ophthalmic surgical apparatus comprises a system for moving 30 the laser beam 8 suited for moving the focal point 6 with respect to an axis 36. For example, the system for moving 30 the laser beam 8 is an opto-mechanical system for moving the beam. More particularly, the system for moving 30 the focal point 6 of the laser beam is configured to constrain the focal point 6 to follow a curvilinear path which has a symmetry of revolution about an axis of rotation 36. Preferably, the surgeon arranges the manual tool 40 in such a way as to align the axis of rotation 36 on the optical axis 21 passing through the centre of the iris 26 and/or of the limbus 7. It is supposed here that the eye 4 remains fixed, without necessarily being immobilised. Particularly advantageously, the path of the focal point 6 of the laser beam 8 is located on the surface of a cylinder or on a helicoid having an axial symmetry, for example with an elliptical or circular section and with determined dimensions or with a determined diameter, with the axis of the cylinder 20 being centred on the iris and/or the limbus.

In an embodiment, the optical system 10 or at least one element of the optical system 10 is mounted on a mobile frame that allows for the rotation of the beam about an axis of rotation with a translation and/or an inclination of the laser beam in relation to this axis of rotation. By aligning the axis of rotation 36 of the laser beam on the optical axis 21 of the crystalline lens, the laser beam 8 makes one rotation about the optical axis 21 of symmetry of the crystalline lens.

By way of example, the system for moving is configured so that the focal point 6 is moved along a circular path with a diameter equal to 4 mm and centred on the optical axis 21. The path of the focal point 6 as such remains in a plane transverse to the axis of rotation 36 of the deflected laser beam. A circular cut of the surface 25 of the anterior capsule of the crystalline lens 5 can as such be carried out. The rotation, at a speed between 30 Hz and 350 Hz is combined with an axial translation, at a speed of movement in z from 100 μm/s to 1250 μm/s. The path of the laser beam as such carries out a helicoid of 200 μm in height over a duration of about 150 ms, with a repetition rate of pulses greater than or equal to 240 kHz, for example 500 kHz.

In this way, during the moving of the focal point 6 of the laser beam 38 along a circular path, the laser beam 8 traverses the plano-concave lens 61 in an annular area located at a constant distance from the optical axis of this plano-concave lens 61. Similarly, the laser beam passes through each interface or optical medium of the anterior segment of the eye at a distance from the optical axis 21 that remains constant, regardless of the focal point 6 over the entire path of the optical beam centred on this optical axis 21. As such, the areas passed through by the laser beam in the various optical components and media are centro-symmetrical with respect to the optical axis 21. The moving of the focal point along a path centred on the optical axis 21 makes it possible to ensure that the focal point 6 has the same geometric optical properties over the entire path. It is as such possible to minimise or even correct the geometric aberrations not only on a focussing point 6, but over an entire curvilinear path centred on the optical axis 21. This specificity makes it possible to obtain a focal spot of a dimension that is very close to the limit of diffraction (typically with a diameter less than 1.2 times the limit of diffraction) while still using a limited numerical aperture while still retaining a focal spot dimension less than 6 μm.

Particularly advantageously, the adaptation interface device 60 comprises at least one annular area, whereon the laser beam 8 is incident, with this annular area contributing to the correction of the geometric optical aberrations on the intraocular focal point 6.

As such, the apparatus is perfectly corrected of the optical aberrations at the focal point 6, over the entire path of the laser beam, with this path being an annular path with a determined diameter.

FIG. 3 shows a system for moving the laser beam according to a first embodiment, based on a rotating prism. A prism 31 is placed, inside the manual tool 40, on the optical path of the laser beam 8. The prism 31 receives an incident laser beam 8 and transmits a deflected laser beam 38. Indeed, the passing through of the prism 31 induces a deviation of the laser beam, with the angle of this deviation being determined by the geometric optical properties of the prism: angle at the top of the prism 31 and refractive index of the material forming this prism 31. The prism 31 is mounted mobile in rotation about an axis of rotation 36, for example on a rotating plate. Preferably, the axis of rotation 36 of the prism is parallel to the optical axis of the incident laser beam 8 on the rotating prism 31. The rotation of the prism 31 about the axis of rotation 36 drives a rotation R of the laser beam 38 deviated by the prism. Consequently, in a plane transversal to the axis of rotation 36, the path 28 of the laser beam 38 deflected by the rotating prism 31 is a circular path about the axis of rotation 36. In a plane transversal to the axis of rotation, the radius of the circular path of the beam is equal to d.

FIG. 4A shows the combination of a system for focussing 10 and of a rotating prism 31. The optical system 10 forms the image of a point source 18 on a focal point 6. By way of example, the optical system 10 comprises two lenses arranged in such a way as to form an afocal optical system. An end of the optical fibre 15, of which the other end is connected to the laser source 1, constitutes for example the point source 18. The afocal system 10 can be configured to produce a determined magnification between the point source 18 and the focal point 6. The rotating prism 31 is arranged between the system for focussing 10 and the focal point 6. The prism 31 results in a deviation of the laser beam 38, and therefore a decentring of the focal point in relation to the optical axis of the incident laser beam on the prism. Consequently, the rotation of the rotating prism 31 about the axis of the laser beam 8 drives a moving of the focal point 6 along a circular path in a plane transverse to the axis of rotation 36 of the prism 31.

FIGS. 4B-4E show in detail the combination of a system for focussing 10 and of a rotating prism 31, in different orientations of the rotating prism 31, in projection in the plane of FIGS. 4B-4E. In FIG. 4B, the angle of rotation of the prism 31 about the axis of rotation 36 is equal to 0 degree, the focal point 6 is located on the plane of FIG. 4B, above the axis of rotation 36. In FIG. 4C, the angle of rotation of the prism 31 about the axis of rotation 36 is equal to 90 degrees, the focal point 6 is located in a plane transverse to the plane of FIG. 4C. In FIG. 4D, the angle of rotation of the prism 31 about the axis of rotation 36 is equal to 135 degrees, the focal point 6 is located in the plane that forms an angle of 135 degrees with the plane of FIG. 4D. In FIG. 4E, the angle of rotation of the prism 31 about the axis of rotation 36 is equal to 180 degrees, the focal point 6 is located in the plane of the FIG. 4E, below the axis of rotation 36. On each FIG. 4B-4E, the deflected laser beam 38 is focussed on a focal point 6 which is moved about the axis of rotation 36, according to the angle of rotation of the rotating prism 31. The rotation R of the rotating prism 31 drives a moving of the focal point 6 in a plane transverse to the axis of rotation 36. Regardless of the angle of rotation of the prism, the focal point 6 remains at a constant distance from the axis 36. In addition with the numerical aperture of the incident beam on the prism and the angle at the top of said prism being low, the geometric aberrations due to the prism remain low and constant along the path 16 which allows for the compensation thereof.

FIG. 5 shows a system for moving the beam according to a second embodiment, based on an opto-mechanical system with a rotating mirror. By way of example, the system for moving of FIG. 5 comprises a system of mirrors comprising a first flat mirror 34 and a second concave mirror 35 of the conical type. The flat mirror 34 is inclined with respect to the optical axis of the incident laser beam, in such a way as to reflect the laser beam 8 to the second concave mirror 35. The second concave mirror 35 reflects the laser beam received from the first mirror 34, and forms a laser beam 38 that is as such offset and/or deviated in relation to the optical axis of the incident laser beam 8. The first mirror 34 is mounted mobile in rotation about an axis of rotation 36, more preferably aligned on the optical axis of the incident laser beam 8 and aligned on the axis of the second conical mirror 35. The rotation of the mirror 34 drives the rotation of the laser beam 38 about the axis of rotation 36. The second mirror 35 reflects the laser beam centro-symmetrically in relation to the axis of rotation 36. As such, the focal point 6 follows a circular path 16 about the axis of rotation 36 with the same rotation speed R as the rotation speed of the first mirror 34.

In the cases shown in FIGS. 3 to 5, the combination of an angular deviation of the laser beam and of a rotation of this deflected laser beam produces a movement of the deflected laser beam 38 along a cone of circular section. The focal point 6 of the pulsed laser beam follows a curvilinear path 16 inside an annular area about the optical axis 21 of the crystalline lens. This annular area is limited by a volume delimited, on the one hand, between two coaxial cones with circular sections and of different diameters, with the axis of these cones being merged and, on the other hand, between two planes transverse to the axis of said cones.

In another embodiment, the system for focussing comprises an off centered aspherical lens. Preferably, the lens of the interface device has a planar surface on the side of the system for focussing. In this case, the geometric aberrations are reduced substantially to the spherical aberration and a negligible residue of off center coma. The aspherical lens of the system for focussing can be configured to perfectly correct these aberrations at any point of the circular path 16 of the focal point.

Particularly advantageously, the combination of a system for focussing comprising an aspherical lens working outside of the axis and a patient interface device comprising a very thick lens having a planar upper surface makes it possible to increase the back focal distance of the system for focussing by about 40% and as such to substantially separate the system for focussing from the eye. Advantageously, this lens can even be diverted off centered with respect to the optical axis of the lens. For example, the off centering of the optical axis of the lens with respect to the axis of rotation of the frame is approximately equal to the radius of the circle that is sought to be described. This element can as such be made to rotate about its geometric centre which then corresponds to the optical axis of the incident beam because it is still the same surface of the lens which is passed through by the incident ray. The ophthalmic surgical instrument obtained as such is very compact and ergonomic. The ophthalmic surgical instrument can as such be used by the surgeon, whether he is right-handed or left-handed, on the right eye as well as on the left eye, by passing over the cheekbone, the brow bone or even above the nose of the patient while still preserving a direct unaltered view, vertically, of the eye of the patient.

The rotation speed of a rotating plate is in general between 10 Hertz and several hundred Hertz. In an embodiment, the rotation speed is equal to 250 Hertz, which makes it possible to carry out one turn in 4 milliseconds.

The apparatus can comprise a device for the synchronised triggering of the emission of the laser pulses and of the system for moving the laser beam. The device for synchronisation can for example be controlled by the operator by means of a pedal.

Alternatively, the rotation of the system for moving 30 is launched at a defined frequency of rotation, for example of a few tens of Hertz. Then, the operator triggers the firing of laser pulses in combination with the rotation of the laser beam.

Advantageously, the optical system for shaping the beam comprises a field diaphragm that determines the numerical aperture of the beam between the system for moving the laser beam and the focal point 6. In practice, the numerical aperture is adjusted between the values of 0.05 and 0.45. As the distance between the focal point and the adaptation interface is less than or equal to about 20 mm, the spatial extent of the laser beam 8 on the optical components of the adaptation interface is limited, which makes it possible to reduce the geometric optical aberrations at the focal point 6.

The ophthalmic surgical apparatus formed as such makes it possible to obtain a focal point 6 that has dimensions close to the limit of diffraction over the entire path 16. It is observed in practice that, over the entire path 16, the laser beam at the focal point 6 is symmetrical with respect to the axis of the adaptation interface device. The size of the beam at the focal point at 1/e² is between a few microns and a few tens of microns according to the numerical aperture chosen. For example, for a numerical aperture of 0.12, the dimension of the focal spot in the eye is approximately 6 micrometers. In order to retain a superposition of the laser impacts in order to ensure a smooth cut, the speed of rotation is chosen at about 100 Hz and the speed of movement in translation parallel to the axis of rotation of 1 mm/s in the eye which makes it possible to limit the total duration of the surgery to about 1 s, which is less than the time characteristic of the movement of a normal eye.

This apparatus makes it possible to make regular circular, continuous, ultra-fast and reproducible cuts. The microscopy analysis shows a cutting quality that is more regular and less rough than that obtained with the current commercial femtosecond lasers. This laser surgical apparatus as such makes it possible to make a circular cut of the anterior capsule of the crystalline lens in a lapse of time with a duration less than one second, even less than a tenth of a second.

A laser ophthalmic surgical apparatus as such dedicated to cutting the rhexis is relatively inexpensive, as it does not require a three-dimensional image acquisition and processing system.

FIGS. 6 to 8 show embodiments of cuts of crystalline lens capsule made on a whole pig eye taken post-mortem.

In the images via binocular microscope of FIGS. 6 to 8, the crystalline lenses were coloured, which has the effect of colouring only the capsule and of increasing the contrast with the other elements of the crystalline lens. In these FIGS. 6-8, the upper portion 25 of the crystalline lens capsule, the inside of the crystalline lens 50 and the rhexis 51 in the central portion are observed. The circles in a dashed line indicate the ideal position of perfect circles that correspond respectively to the cutting of the capsule 150, the cutting of the crystalline lens 250 and the edges of the rhexis 350.

The cuts shown in FIGS. 6 to 8 were then dehydrated in order to be observed under the binocular microscope. It is observed that the cuts respond to the precision, reproducibility and quality criteria sought. The difference between the actual cut and a perfect circle is low. Even in the case where the cuts are not perfectly circular (FIG. 9), the cuts are extremely regular. The cuts of the capsule of the crystalline lens are continuous and do not have any apparent shearing.

However the shearing of the capsulorhexis are known to be at the origin of a large portion of the immediate or later complications of this type of surgery. The shearing of the capsulorhexis can have very harmful consequences on the extraction of the crystalline lens or the installation of the intraocular implant and it stability over time.

At high magnification (×1000), in FIG. 8, the edges 150 of the capsule and the section 250 cut in the thickness of the crystalline lens are observed.

These cuts 150, 250 are of excellent quality and do not have any shearing. The cuts are regular and globally very smooth. Even at high magnification, no roughness that could be due to a postage stamp laser cutting effect is observed, contrary to what has often been observed for a cut with a femtosecond laser.

In certain cases, a few surface irregularities are observed. The section of the cut remains however of very good quality in the thickness of the capsule. Sometimes, the rhexis can appear to still be attached however a very slight traction by means of forceps makes it possible to easily extract this rhexis.

Various tests have been conducted, with a rate of laser firings of 100 kHz and a rotation speed of the laser beam of 40 Hz for example. The rate of coverage is defined as the ratio between the intersection section between two adjacent laser impacts and the impact surface of one of these laser firings. The rate of coverage in particular depends on the impact surface of a laser firing, on the repetition rate of the laser pulses and on the rotation speed of the moving of the laser beam. Even with a rate of coverage less than about 50%, the cut remains continuous and regular.

The current understanding of these results is that the duration of the picosecond or nanosecond pulses makes it possible to capitalise simultaneously on the mechanical effects of disruption, linked to the energy of the laser pulses, and on the highly localised low thermal effects, linked to the thermal depositing of these laser pulses. On the contrary, femtosecond pulses produce only disruption effects, which would explain the irregular edges produced by a cut via a femtosecond laser. However, the thermal effects remain sufficiently limited to not damage the ocular tissues located around the cut. Preferably the laser source is configured to produce a low but non-negligible portion (typically 5 to 40%) of its energy in a time support with a duration between 50 ps and 500 ps. Advantageously the laser source produces pulses of which 60% to 90% of the energy is within a time profile with a duration less than 5 ps and of which the rest of the energy is spread out according to a roughly Gaussian profile over a duration between 50 ps and 100 ps.

Reproducibility tests have been conducted on many test samples, taken post-mortem from animals.

The results obtained for the cutting of the crystalline lens capsule are of excellent quality. A cutting effect is obtained that has edges that are practically as regular as a manual cut, with the cut being curvilinear, with a constant or quasi-constant radius of curvature over the entire path and aesthetically comparable with a manual cut and therefore more regular than cuts obtained by femtosecond laser. In addition, the cut has circularity advantages that are similar to those obtained with a femtosecond laser.

The cut is fast and can be completed in a duration between 150 ms to a few hundred milliseconds.

The device does not require a costly and time-consuming three-dimensional imaging system. As such, the surgery is faster than with a femtosecond laser surgical apparatus.

Other applications of this picosecond or nanosecond laser device for ophthalmic surgery are considered for surgery of the anterior segment of the eye. In particular, this laser apparatus can have applications for surgery on the cornea aiming to correct presbyopia, astigmatism or even in grafting or implanting operations of intra-corneal rings.

Using a nanosecond or picosecond laser source substantially reduces the cost of the source. On the other hand, nanosecond or picosecond laser technologies are in addition proven and integrated and therefore generally increasingly robust.

On the other hand, using a nanosecond or picosecond laser source is compatible with a fibre optic output, contrary to a fs laser. Using a fibre optic laser source makes it possible to improve the spatial quality of the laser beam. In addition, using a fibre optic laser source makes it possible to propose a compact and flexible apparatus.

Adjusting the speed of movement of the laser beam according to the repetition rate of the laser and the duration of the laser pulses makes it possible to provide good coverage of the focussed laser spots, and as such obtain a continuous cut, without shearing.

1-12. (canceled)

13. Ophthalmic surgical apparatus (100) comprising:
a laser source (1) suitable for emitting a beam of laser pulses (8);
an optical focussing system (10, 20) for focussing the pulsed laser beam (8) on a focal point (6) of the anterior segment of an eye (4); and
a system (30) for moving the pulsed laser beam configured to move the focal point (6) along a predetermined path (16);
wherein:
the laser source (1) generates a pulsed laser beam (8) that has a duration of about one picosecond to one nanosecond;
the optical focussing system (10, 20) is configured to focus the pulsed laser beam (8) on a focal point (6) in the vicinity of a surface (25) of the anterior segment of the eye (4), the focal point (6) being located at a distance d other than zero from an optical axis (21) of symmetry of the anterior segment of the eye (4);
the system (30) for moving the beam of laser pulses includes a single degree of freedom of rotation about an axis of rotation (36) so as to move said focal point (6) along a curved path (16) located in an annular area about the optical axis (21) of symmetry of the anterior segment of the eye (4); and
the optical focussing system (10, 20) being configured so as to limit the geometric optical aberrations at the focal point (6) and on the entire curved path (16) in said annular area about the optical axis (21) of symmetry of the anterior segment of the eye (4).

14. Ophthalmic surgical apparatus (100) according to claim 13 wherein the system (30) for moving the pulsed laser beam comprises an optical system (31, 34, 35) arranged on an optical path of the laser beam (8), with the optical system (31, 34, 35) being suited to receive the incident laser beam (8) and configured to form an angularly deflected or translated laser beam (38) with respect to the incident laser beam (8), and wherein said optical system (31, 34, 35) comprises at least one optical component (31, 34) mounted mobile in rotation about said axis of rotation (36) in such a way as to produce a rotation of the laser beam (38).

15. Ophthalmic surgical apparatus (100) according to claim 14 wherein the system (30) for moving the pulsed laser beam comprises a prism (31) arranged on an optical path of the pulsed laser beam (8), with said prism (31) being mounted mobile in rotation about an axis of rotation (36).

16. Ophthalmic surgical apparatus (100) according to claim 14 wherein the system (30) for moving the pulsed laser beam (8) comprises at least one mirror (34) arranged on an optical path of the pulsed laser beam (8), in such a way as to induce an angular deviation and/or a lateral shift of the pulsed laser beam (8), and said at least one mirror (34) being mounted in rotation about an axis of rotation (36).

17. Ophthalmic surgical apparatus (100) according to claim 13, wherein the system (30) for moving the pulsed laser beam (8) is configured to move said focal point (6) along a circular path (16) with a determined radius.

18. Ophthalmic surgical apparatus (100) according to claim 13, wherein the system (30) for moving the pulsed laser beam further comprises a degree of freedom of translation along an axis of translation parallel to the axis of rotation (36), and wherein the system for moving is configured to move said focal point (6) along a helical path with a circular section and determined radius.

19. Ophthalmic surgical apparatus (100) according to claim 13, comprising, on the one hand, a manual tool (40) that comprises the optical focussing system (10, 20) and the system (30) for moving the pulsed laser beam, and, on the other hand, a fibre optic link (15) arranged between the laser source (1) and the manual tool (40).

20. Ophthalmic surgical apparatus (100) according to claim 14, comprising, on the one hand, a manual tool (40) that comprises the optical focussing system (10, 20) and the system (30) for moving the pulsed laser beam, and, on the other hand, a fibre optic link (15) arranged between the laser source (1) and the manual tool (40).

21. Ophthalmic surgical apparatus (100) according to claim 17, comprising, on the one hand, a manual tool (40) that comprises the optical focussing system (10, 20) and the system (30) for moving the pulsed laser beam, and, on the other hand, a fibre optic link (15) arranged between the laser source (1) and the manual tool (40).

22. Ophthalmic surgical apparatus (100) according to claim 18, comprising, on the one hand, a manual tool (40) that comprises the optical focussing system (10, 20) and the system (30) for moving the pulsed laser beam, and, on the other hand, a fibre optic link (15) arranged between the laser source (1) and the manual tool (40).

23. Ophthalmic surgical apparatus (100) according to claim 19, wherein the manual tool (40) comprises a semi-reflective mirror or a dichroic mirror arranged on the optical path of the laser beam (8) and wherein the manual tool (40) is adapted to optically combine a binocular microscope in such a way as to provide a visual control in real time of the anterior segment of the eye (4).

24. Ophthalmic surgical apparatus (100) according to claim 13, further comprising an adaptation interface device (60) that comprises a plate with planar and parallel faces and/or a plano-concave plate (61), with the adaptation interface device (60) having at least one optical surface configured in such a way as to correct the optical aberrations at the focal point (6) and over said path (16) of said focal point (6).

25. Ophthalmic surgical apparatus (100) according to claim 14, further comprising an adaptation interface device (60) that comprises a plate with planar and parallel faces and/or a plano-concave plate (61), with the adaptation interface device (60) having at least one optical surface configured in such a way as to correct the optical aberrations at the focal point (6) and over said path (16) of said focal point (6).

26. Ophthalmic surgical apparatus (100) according to claim 17, further comprising an adaptation interface device (60) that comprises a plate with planar and parallel faces and/or a plano-concave plate (61), with the adaptation interface device (60) having at least one optical surface configured in such a way as to correct the optical aberrations at the focal point (6) and over said path (16) of said focal point (6).

27. Ophthalmic surgical apparatus (100) according to claim 18, further comprising an adaptation interface device (60) that comprises a plate with planar and parallel faces and/or a plano-concave plate (61), with the adaptation interface device (60) having at least one optical surface configured in such a way as to correct the optical aberrations at the focal point (6) and over said path (16) of said focal point (6).

28. Ophthalmic surgical apparatus (100) according to claim 19, further comprising an adaptation interface device (60) that comprises a plate with planar and parallel faces and/or a plano-concave plate (61), with the adaptation interface device (60) having at least one optical surface configured in such a way as to correct the optical aberrations at the focal point (6) and over said path (16) of said focal point (6).

29. Ophthalmic surgical apparatus (100) according to claim 13, further comprising a device for triggering laser firings of the laser source (1) and of the system for moving (60) the pulsed laser beam.

30. Ophthalmic surgical apparatus (100) according to claim 13, wherein the laser source (1) emits laser pulses at a wavelength between 700 nm and 1350 nm, preferably between 1025 nm and 1080 nm.

31. Ophthalmic surgical apparatus (100) according to claim 13, wherein the laser source (1) emits laser pulses at a repetition rate between 20 kHz and 1 MHz.
